(19) United States
(12) Patent Application Publication
(10) Pub. No.: US 2021/0093895 A1
(43) Pub. Date: Apr. 1, 2021

(54) METHOD AND SYSTEM FOR SUPPORTING SKIN TREATMENT USING ULTRASONIC WAVES, AND NON-TEMPORARY COMPUTER-READABLE RECORDING MEDIUM

(71) Applicant: REZIENA INC., Seoul (KR)

(72) Inventors: Tae Kyun KIM, Gyeonggi-do (KR); Seung Woo SHIN, Gyeonggi-do (KR)

(21) Appl. No.: 16/985,740

(22) Filed: Aug. 5, 2020

Related U.S. Application Data
(63) Continuation of application No. PCT/KR2019/001420, filed on Feb. 1, 2019.

(30) Foreign Application Priority Data
Feb. 6, 2018 (KR) 10-2018-0014795

(51) Int. Cl. A61N 7/00

(52) U.S. Cl. CPC A61N 7/00; A61N 2007/0052; A61N 2007/0078; A61N 2007/0034

(57) ABSTRACT

According to an embodiment of the present disclosure, a system for supporting a skin treatment using ultrasound comprises a treatment map management unit configured to determine an ultrasonic treatment map of at least one of a treatment intensity, a treatment number and a treatment depth for at least one skin section of a user with reference to skin condition information including information about at least one of moisture content and wrinkles of the user's skin; a guide providing unit configured to provide the user with a guide for treatment based on the determined ultrasonic treatment map; and a monitoring management unit configured to monitor whether the user follows the guide with reference to treatment action information of the user according to the provided guide.

### TECHNICAL FIELD

The present disclosure relates to a method and a system for supporting skin treatment using ultrasound, and a non-transitory computer-readable storage medium.

### BACKGROUND

As interest in appearance increases, the market for medical devices related to skin care is growing. In particular, non-invasive medical devices have received a lot of attention due to the safety in treatment and the patient's rejection to invasive medical devices. Among the existing non-invasive medical skin treatment devices, medical skin treatment devices using high-intensity focused ultrasound (HIFU) are known to cause the least damage to the surface of the skin and have excellent skin improvement effect, and research and development related thereto is more actively conducted.

In this regard, as an example of the prior art, a method and an apparatus for high-intensity focused ultrasound have been suggested. In this case, a transducer is divided into a plurality of sections to generate electrode array effects and to be combined with transducers having different resonance frequencies to have a plurality of resonant frequencies and a plurality of treatment depths, and a plurality of high-intensity focused ultrasounds is irradiated in various patterns from a handpiece to a treatment site to generate a high-intensity focused thermal coagulation point in the target skin layer.

However, according to the above-described prior art and other techniques so far suggested, the intensity and penetration depth of ultrasonic energy which have been clinically proven depending on the skin condition need to be determined, but it is not only dangerous to determine and use but also difficult to operate by an ordinary person who is not a professionally trained operator (for example, a doctor).

Accordingly, the present inventors provide a customized ultrasonic skin treatment and a guide to the treatment based on the skin condition of a user and provide a technology that supports anyone to easily and safely perform an ultrasonic skin treatment by monitoring whether the guide is followed.

### DISCLOSURE OF THE INVENTION

### Problems to be Solved by the Invention

The present disclosure has been made to solve the above-described problems of the prior art.

Also, the present disclosure has been made to provide a skin treatment solution which is personalized for a user based on the user's skin condition information, such as moisture content, wrinkles and the like.

Further, the present disclosure has been made to support anyone to easily perform a skin treatment using ultrasound.

Furthermore, the present disclosure has been made to provide a guide for proper use and prevention of safety problems and improper use which may occur during a skin treatment.

### Means for Solving the Problems

To this end, representative structures of the present disclosure are described as follows.

According to an embodiment of the present disclosure, a system for supporting a skin treatment using ultrasound comprises, a treatment map management unit configured to determine an ultrasonic treatment map of at least one of a treatment intensity, a treatment number and a treatment depth for at least one skin section of a user with reference to skin condition information including information about at least one of moisture content and wrinkles of the user's skin; a guide providing unit configured to provide the user with a guide for treatment based on the determined ultrasonic treatment map; and a monitoring management unit configured to monitor whether the user follows the guide with reference to treatment action information of the user according to the provided guide.

According to another embodiment of the present disclosure, A method for supporting a skin treatment using ultrasound comprises, determining an ultrasonic treatment map of at least one of a treatment intensity, a treatment number and a treatment depth for at least one skin section of a user with reference to skin condition information including information about at least one of moisture content and wrinkles of the user's skin; providing the user with a guide for treatment based on the determined ultrasonic treatment map; and monitoring whether the user follows the guide with reference to treatment action information of the user according to the provided guide.

There are further provided other methods and other systems for implementing the present disclosure and a non-transitory computer-readable storage medium that stores a computer program for executing the above-described method.

### Effects of the Invention

According to the present disclosure, it is possible to provide a skin treatment solution which is personalized for a user based on the user's skin condition information, such as moisture content, wrinkles and the like.

Further, according to the present disclosure, it is possible to support anyone to easily perform a skin treatment using ultrasound.

Furthermore, according to the present disclosure, it is possible to provide a guide for proper use and prevention of safety problems and improper use which may occur during a skin treatment.

### BRIEF DESCRIPTION OF THE DRAWINGS

FIG. 1 is an illustration of a schematic configuration of the entire system that supports a skin treatment using ultrasound according to an embodiment of the present disclosure.

FIG. 2 is a detailed view illustrating an internal configuration of a skin treatment support system according to an embodiment of the present disclosure.

FIG. 3 to FIG. 6 illustrate examples of a process for determining an ultrasonic treatment map according to an embodiment of the present disclosure.

FIG. 7 illustrates examples of a process for supporting an ultrasonic skin treatment according to an embodiment of the present disclosure.

### BEST MODE FOR CARRYING OUT THE INVENTION

Detailed descriptions of the present disclosure will be made with reference to the accompanying drawings illustrating specific embodiments of the present disclosure as examples. These embodiments will be described in detail such that the present disclosure can be performed by a person with ordinary skill in the art. It should be understood that various embodiments of the present disclosure are different, but are not necessarily mutually exclusive. For example, a specific shape, structure, and characteristic of an embodiment described herein may be implemented in another embodiment without departing from the spirit and scope of the present disclosure. In addition, it should be understood that a position or an arrangement of each component in each embodiment may be changed without departing from the spirit and scope of the present disclosure. Accordingly, there is no intent to limit the present disclosure to the detailed descriptions to be described below, and the scope of the present disclosure is defined by the appended claims and encompasses all equivalents that fall within the scope of the appended claims. Like reference numerals in the drawings refer to the same or like components throughout various aspects.

Hereinafter, in order to easily perform the invention by a person with ordinary skill in the art, exemplary embodiments of the present disclosure will be described in detail with reference to the accompanying drawings.

Configuration of Entire System

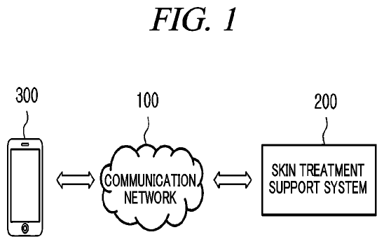

FIG. 1 is an illustration of a schematic configuration of the entire system that supports a skin treatment using ultrasound according to an embodiment of the present disclosure.

As illustrated in FIG. 1, the entire system according to an embodiment of the present disclosure may include a communication network 100, a skin treatment support system 200 and a user device 300.

First, the communication network 100 according to an embodiment of the present disclosure may be configured without regard to communication modes such as wired communication and wireless communication, and may be configured as various communication networks, such as a LAN (Local Area Network), a MAN (Metropolitan Area Network), WAN (Wide Area Network) and the like. Desirably, the communication network 100 referred to herein may be the well-known Internet or World Wide Web (WWW). However, the communication network 100 may include, at least in part, a known wire/wireless data communication network, a known telephone network or a known wire/wireless television communication network, without being limited thereto.

For example, the communication network 100 may be a wireless communication network and may implement, at least in part, conventional communication methods, such as WiFi communication, WiFi-Direct communication, LTE (Long Term Evolution) communication, Bluetooth communication (for example, BLE (Bluetooth Low Energy) communication), infrared ray communication, ultrasonic communication and the like.

The skin treatment support system 200 according to an embodiment of the present disclosure may communicate with the user device 300, which will be described later, through the communication network 100, determine an ultrasonic treatment map of at least one of a treatment intensity, a treatment number and a treatment depth for at least one skin section of a user with reference to skin condition information including information about at least one of moisture content and wrinkles of the user's skin, provide the user with a guide for treatment based on the determined ultrasonic treatment map, and monitor whether the user follows the guide with reference to treatment action information of the user according to the provided guide.

Also, according to an embodiment of the present disclosure, the skin treatment support system 200 may be implemented on a conventional system that performs a skin treatment using ultrasound (specifically, high-intensity focused ultrasound (HIFU)) or may include at least some of the features (for example, a probe that irradiates ultrasonic energy, a cartridge, etc.) of the conventional system.

Further, according to an embodiment of the present disclosure, the skin treatment support system 200 may further include an image sensing module (for example, a camera module), a moisture sensing module (for example, an electric conductivity sensor) and the like for measuring the skin condition information including information about at least one of moisture content and wrinkles of the skin.

The components and functions of the skin treatment support system 200 according to an embodiment of the present disclosure will be described in detail below. Meanwhile, the above description of the skin treatment support system 200 is provided for the purpose of illustration, but it will be obvious to a person with ordinary skill in the art that at least some of the functions or components required for the skin treatment support system 200 may be implemented inside the user device 300, which will be described later, or an external system (not illustrated) or included in the user device 300 or the external system (not illustrated).

According to an embodiment of the present disclosure, the user device 300 is a digital device having a function to access and communicate with the skin treatment support system 200 through the communication network 100, and any portable digital device equipped with memory means and a microprocessor and thus having operating capabilities, such as a smart phone, a tablet PC and the like may be adopted as the user device 300 according to the present disclosure.

Meanwhile, according to an embodiment of the present disclosure, the user device 300 may include an application for supporting the function to treat the skin using ultrasound according to the present disclosure. The application may be downloaded from the skin treatment support system 200 or an external application distribution server (not illustrated).

Configuration of Skin Treatment Support System

Hereinafter, an internal configuration of the skin treatment support system 200 that performs an important function for implementing the present disclosure and functions of the respective components will be described.

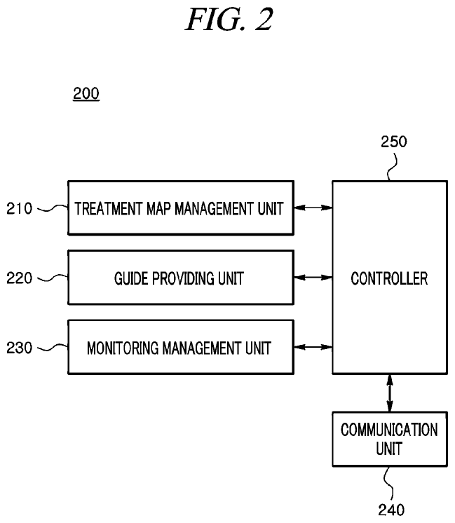

FIG. 2 is a detailed view illustrating the internal configuration of the skin treatment support system 200 according to an embodiment of the present disclosure.

The skin treatment support system 200 according to an embodiment of the present disclosure may be a digital device equipped with memory means and a microprocessor and thus having operating capabilities. As illustrated in FIG. 2, the skin treatment support system 200 may be configured including a treatment map management unit 210, a guide providing unit 220, a monitoring management unit 230, a communication unit 240, and a controller 250. According to an embodiment of the present disclosure, in the skin treatment support system 200, at least some of the treatment map management unit 210, the guide providing unit 220, the monitoring management unit 230, the communication unit 240, and the controller 250 may be program modules that communicate with an external system. These program modules may be included in the skin treatment support system 200 in the form of an operating system, an application program, or other program modules and may be physically stored in a variety of well-known memory devices. In addition, these program modules may be stored in a remote memory device capable of communicating with the skin treatment support system 200. Such program modules comprehensively include routines, subroutines, programs, objects, components, data structures and the like for performing a specific task or executing a specific abstract data type, which will be described later, but may not be limited thereto.

First, the treatment map management unit 210 according to an embodiment of the present disclosure may determine an ultrasonic treatment map of at least one of a treatment intensity (for example, the intensity of ultrasonic energy, such as 0.15 J (Joule)), a treatment number and a treatment depth (for example, 1.5 mm or 3 mm) for at least one skin section of a user with reference to skin condition information including information about at least one of moisture content and wrinkles of the user's skin. According to an embodiment of the present disclosure, the ultrasonic treatment map may define the user's skin, which is the target of an ultrasonic treatment, into at least one section having a predetermined size and show the result of a match between each section and at least one of the treatment intensity, the treatment number and the treatment depth (such as a map, a relationship diagram or the like).

For example, the treatment map management unit 210 according to an embodiment of the present disclosure may determine a treatment level for at least one skin section of the user based on the distribution (or amount) and size of wrinkles of the user's skin.

More specifically, the treatment map management unit 210 according to an embodiment of the present disclosure may calculate a wrinkle evaluation score for determining the degree of wrinkles of the user based on the distribution (or amount) and size of wrinkles of the user's skin and determine a treatment level (or a treatment mode) for at least one skin section of the user corresponding to the calculated wrinkle evaluation score.

Further, the treatment map management unit 210 according to an embodiment of the present disclosure may refer to the determined treatment level to determine a treatment depth and a treatment intensity for at least one skin section corresponding to the treatment level.

Meanwhile, to determine the treatment depth or the treatment intensity, the treatment map management unit 210 according to an embodiment of the present disclosure may further refer to a lookup table for at least one treatment depth (or at least one treatment intensity) corresponding to at least one treatment level.

Also, the treatment map management unit 210 according to an embodiment of the present disclosure may correct the determined treatment depth or treatment intensity based on the moisture content of the user's skin if necessary.

More specifically, if the treatment map management unit 210 according to an embodiment of the present disclosure determines a treatment level for at least one skin section of the user based on the distribution (or amount) and size of wrinkles of the user's skin, the treatment map management unit 210 may correct the treatment depth and the treatment intensity for at least one skin section corresponding to the determined treatment level (for example, increase the treatment intensity as the skin is drier and decrease the treatment intensity as the skin is more moisturized) with reference to the moisture content of the user's skin.

Meanwhile, according to an embodiment of the present disclosure, the treatment map management unit 210 according to an embodiment of the present disclosure may refer to the relationship between a predetermined moisture content of the skin and a predetermined attenuation of ultrasonic energy in order to perform the above-described correction.

For example, according to an embodiment of the present disclosure, the treatment map management unit 210 according to an embodiment of the present disclosure may determine the degree of correction of the treatment intensity of the user's skin with reference to the ultrasonic energy attenuation corresponding to the age and moisture content of the user and correct the treatment intensity for at least one skin section corresponding to the treatment level based on the determined degree of correction.

Meanwhile, according to an embodiment of the present disclosure, the ultrasonic energy attenuation relationship may be determined by a patterned result based on age, sex, race, region and the like of a plurality of users.

Further, according to an embodiment of the present disclosure, the treatment map management unit 210 may further refer to an ultrasonic treatment reference map related to profile information of the user to determine the ultrasonic treatment map. According to an embodiment of the present disclosure, the profile information may include age, sex, skin type (for example, oily skin type, dry skin type, etc.) and the like of the user.

For example, according to an embodiment of the present disclosure, the treatment map management unit 210 specifies an ultrasonic treatment reference map corresponding to the profile information of the user and refers to the user's skin condition information (for example, distribution (or amount) of wrinkles, size of wrinkles, moisture content, etc.) to correct at least one of the treatment intensity, the treatment number and the treatment depth for at least one skin section specified by the ultrasonic treatment reference map and thus to determine an ultrasonic treatment map of at least one of the treatment intensity, the treatment number and the treatment depth for at least one skin section of the user.

More specifically, the treatment map management unit 210 according to an embodiment of the present disclosure may determine the treatment number depending on the treatment depth for at least one skin section specified by the ultrasonic treatment reference map corresponding to the profile information of the user as the treatment number depending on the treatment depth for at least one skin section of the user. Further, the treatment map management unit 210 according to an embodiment of the present disclosure may determine a treatment level for at least one skin section of the user based on the distribution (or amount) and size of wrinkles of the user's skin and refer to the moisture content of the user's skin to determine a first treatment depth and a first treatment intensity for at least one skin section depending on the determined treatment level. Furthermore, the skin treatment support system 200 according to an embodiment of the present disclosure may refer to the first treatment depth and the first treatment intensity to correct a second treatment depth and a second treatment intensity specified by the ultrasonic treatment reference map and thus to determine a treatment intensity depending on the treatment depth for at least one skin section of the user. Besides, the treatment map management unit 210 according to an embodiment of the present disclosure may refer to the determined treatment depth, treatment intensity and treatment number to determine an ultrasonic treatment map for the user.

Then, according to an embodiment of the present disclosure, the guide providing unit 220 may provide the user with a guide for treatment based on the ultrasonic treatment map determined by the treatment map management unit 210.

Specifically, the guide providing unit 220 according to an embodiment of the present disclosure may refer to the ultrasonic treatment map for the user to provide a guide including information about at least one of what the user needs to do, depending on at least one skin section of the user in need of a skin treatment, a treatment intensity and a treatment section, and the progress of a skin treatment. The guide providing unit 220 according to an embodiment of the present disclosure may provide the guide through the user device 300.

Then, according to an embodiment of the present disclosure, the monitoring management unit 230 may perform a function to monitor whether the user follows the guide with reference to treatment action information of the user according to the provided guide. According to an embodiment of the present disclosure, the treatment action information may include information about actions estimated to be performed by the user for treatment while the guide is provided.

For example, according to an embodiment of the present disclosure, the monitoring management unit 230 may estimate treatment action information for the user from information about at least one of a treatment action image of the user, a treatment direction of the user and the presence or absence of contact with the user's skin (i.e., the presence or absence of contact between the user's skin and the skin treatment support system 200 or an external system (for example, an ultrasonic irradiation probe or the like)). The monitoring management unit 230 may confirm whether the user follows the guide by comparing and analyzing whether the estimated treatment action information matches with at least one of a reference treatment action, a reference treatment position and a reference treatment section according to the guide.

More specifically, when the monitoring management unit 230 according to an embodiment of the present disclosure confirms that the skin treatment support system 200 is in contact with the user's skin, the monitoring management unit 230 may determine a posture of the user based on the treatment action image or treatment direction of the user, estimate treatment action information of the user based on the determined posture of the user and confirm whether the user follows the guide by comparing and analyzing whether the estimated treatment action information matches with a reference treatment action, a reference treatment position or a reference treatment section according to the guide.

Meanwhile, according to an embodiment of the present disclosure, the monitoring management unit 230 may obtain information about a treatment action image of the user through the user device 300 and obtain information about a treatment direction of the user from an acceleration sensor, a gravity sensor, a Gyro sensor and the like which may be included in the skin treatment support system 200 or an external system (not illustrated; for example, a separate device including the sensor). Also, the monitoring management unit 230 may obtain information about the presence or absence of contact with the user's skin from an electric conductivity sensor which may be included in the skin treatment support system 200 or an external system (not illustrated; for example, a separate device including the sensor).

Meanwhile, when the monitoring management unit 230 according to an embodiment of the present disclosure determines that the user does not follow the guide, the monitoring management unit 230 may provide a warning message or a correction guide to the user or stop an operation for treatment (for example, irradiation of ultrasonic energy for skin treatment). Further, if the operation for treatment is stopped, the monitoring management unit 230 according to an embodiment of the present disclosure may restart the stopped operation for treatment when treatment action information of the user for which confirmation by the user is input or which is reestimated matches with at least one of a reference treatment action, a reference treatment position and a reference treatment section according to the guide provided to the user.

Then, the communication unit 240 according to an embodiment of the present disclosure may perform a function to enable data transmission and reception to/from the treatment map management unit 210, the guide providing unit 220 and the monitoring management unit 230.

Finally, the controller 250 according to an embodiment of the present disclosure may perform a function to control data flow among the treatment map management unit 210, the guide providing unit 220, the monitoring management unit 230 and the communication unit 240. That is, the controller 250 according to the present disclosure may control data flow from/to the outside of the skin treatment support system 200 or data flow among the components of the skin treatment support system 200 to control the treatment map management unit 210, the guide providing unit 220, the monitoring management unit 230 and the communication unit 240 to perform their own functions, respectively.

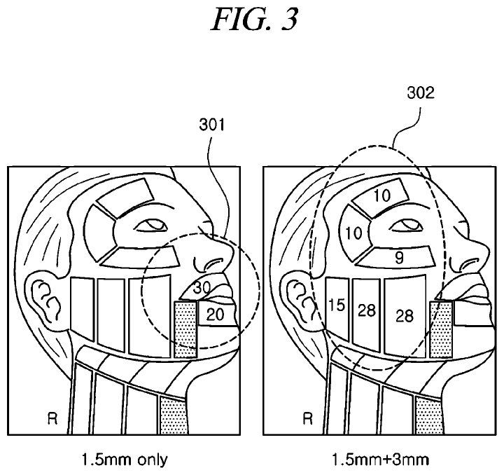

FIG. 3 to FIG. 6 illustrate examples of a process for determining an ultrasonic treatment map according to an embodiment of the present disclosure.

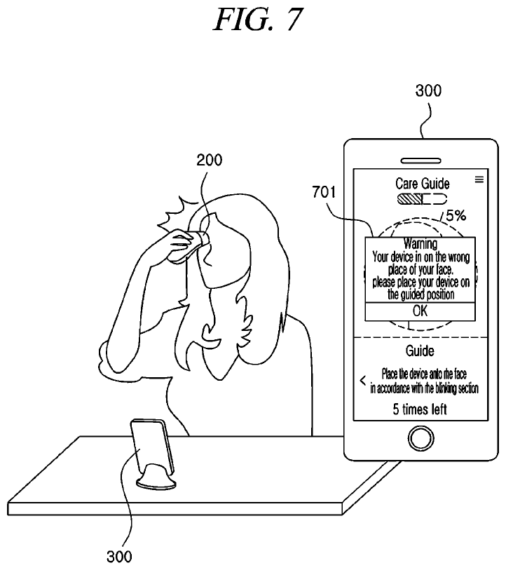

FIG. 7 illustrates examples of a process for supporting an ultrasonic skin treatment according to an embodiment of the present disclosure.

According to an embodiment of the present disclosure, it can be assumed that the skin treatment support system 200 is implemented on a conventional system that performs a skin treatment using ultrasound (specifically, high-intensity focused ultrasound (HIFU)).

First, the skin treatment support system 200 according to an embodiment of the present disclosure may obtain skin condition information including information about at least one of moisture content and wrinkles of the user's skin.

Then, the skin treatment support system 200 according to an embodiment of the present disclosure may determine an ultrasonic treatment map of at least one of a treatment intensity, a treatment number and a treatment depth for at least one skin section of the user with reference to skin condition information including information about at least one of moisture content and wrinkles of the user's skin.

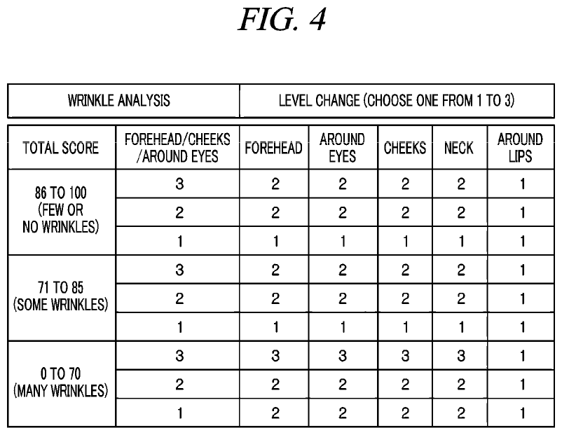

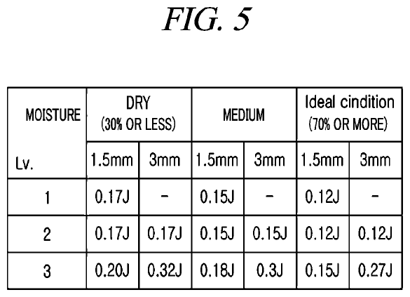

For example, referring to FIG. 3, the skin treatment support system 200 according to an embodiment of the present disclosure may specify an ultrasonic treatment reference map corresponding to profile information of the user and determine treatment numbers 301 and 302 depending on a treatment depth for at least one skin section specified by the ultrasonic treatment reference map. Also, referring to FIG. 4 and FIG. 5, the skin treatment support system 200 according to an embodiment of the present disclosure may determine a treatment level for at least one skin section of the user based on the distribution (or amount) and size of wrinkles of the user's skin and refer to the moisture content of the user's skin to determine a first treatment depth and a first treatment intensity for at least one skin section depending on the determined treatment level. Then, the skin treatment support system 200 according to an embodiment of the present disclosure may refer to the first treatment depth and the first treatment intensity to correct a second treatment depth and a second treatment intensity specified by the ultrasonic treatment reference map and thus to determine a treatment intensity depending on the treatment depth for at least one skin section of the user. According to an embodiment of the present disclosure, an ultrasonic treatment map for the user may be determined based on at least one of the determined treatment intensity, treatment depth and treatment intensity for at least one skin section.

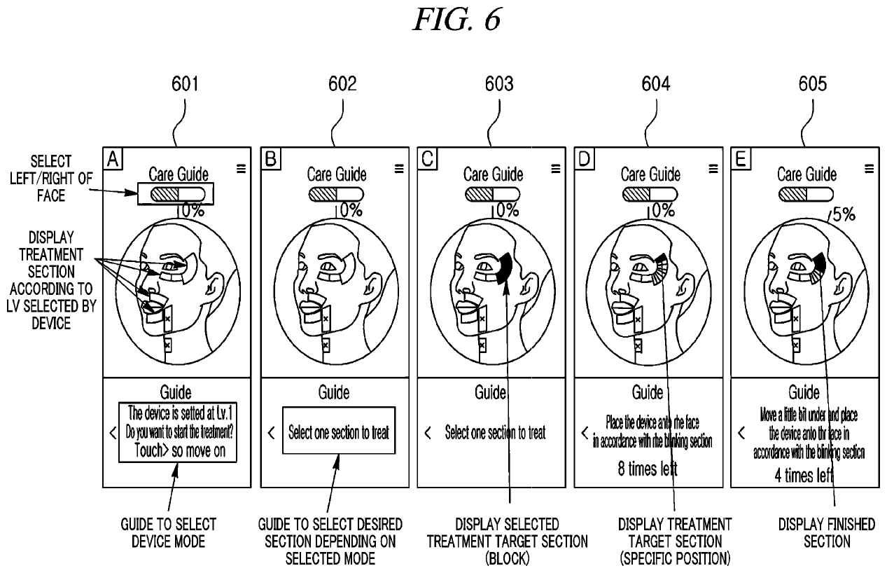

Then, referring to FIG. 6, the skin treatment support system 200 according to an embodiment of the present disclosure may provide the user with guides 601, 602, 603, 604 and 605 based on the ultrasonic treatment map. According to an embodiment of the present disclosure, the guide may be provided through the user device 300.

Then, when the user follows the guide, the skin treatment support system 200 according to an embodiment of the present disclosure may enable ultrasonic energy corresponding to the ultrasonic treatment map to be irradiated onto the user's skin. Meanwhile, referring to FIG. 7, if it is determined that the user does not follow the guide, the skin treatment support system 200 according to an embodiment of the present disclosure may provide a warning message 701 to the user and stop the treatment.

Meanwhile, according to an embodiment of the present disclosure, the skin treatment support system 200 may support multi-depth caring to irradiate ultrasonic energy depending on the treatment depth without changing or replacing a probe even if the treatment depth (for example, 1.5 mm, 3 mm) changes variously during the skin treatment. That is, it is possible to improve the convenience in manipulation for skin treatment and reduce a treatment time.

The above-described embodiments of the present disclosure may be implemented in the form of a program command that can be performed through various computer components, and may be recorded on a computer-readable storage medium. The computer-readable storage medium may include a program command, a data file, and a data structure individually or a combination thereof. The program command recorded in the computer-readable storage medium may be specially designed and configured for the present disclosure, or may be known to those skilled in a computer software field to be used. Examples of the computer-readable storage medium include a magnetic medium such as a hard disk, a floppy disk, and a magnetic tape, an optical medium such as a CD-ROM and a DVD, a magneto-optical medium such as a floptical disk, and a hardware device such as a ROM, a RAM, and a flash memory, that are specially configured to store and perform program commands. Examples of the program commands may include a machine code generated by a compiler and a high-level language code that can be executed in a computer using an interpreter. The hardware device may be configured as at least one software-module in order to perform the operations of the present disclosure, and vice versa.

While the present disclosure has been described with reference to specific details, such as detailed components, specific embodiments, and drawings, these are only examples to facilitate the overall understanding of the present disclosure and the present disclosure is not limited thereto. It will be understood by a person with ordinary skill in the art that various modifications and changes can be made from the descriptions above.

Therefore, the spirit of the present disclosure is defined not by the above-described embodiments, but by the appended claims, and encompasses all modifications and equivalents that fall within the scope of the appended claims.

We claim:

1. A system for supporting a skin treatment using ultrasound, comprising:
a treatment map management unit configured to determine an ultrasonic treatment map of at least one of a treatment intensity, a treatment number and a treatment depth for at least one skin section of a user with reference to skin condition information including information about at least one of moisture content and wrinkles of the user's skin;
a guide providing unit configured to provide the user with a guide for treatment based on the determined ultrasonic treatment map; and
a monitoring management unit configured to monitor whether the user follows the guide with reference to treatment action information of the user according to the provided guide.

2. The system of claim 1,
wherein the treatment map management unit further refers to an ultrasonic treatment reference map related to profile information of the user to determine the ultrasonic treatment map.

3. The system of claim 2,
wherein the treatment map management unit specifies a treatment number for at least one skin section of the user based on the ultrasonic treatment reference map.

4. The system of claim 1,
wherein the treatment map management unit determines a treatment level for at least one skin section of the user based on the distribution and size of wrinkles of the user's skin and determines a treatment depth and a treatment intensity for at least one skin section depending on the determined treatment level with reference to the moisture content of the user's skin.

5. The system of claim 1,
wherein when the monitoring management unit determines that the user does not follow the provided guide, the monitoring management unit provides a warning message or a correction guide to the user or stop an operation for the treatment.

6. The system of claim 1,
wherein the monitoring management unit senses whether the user follows the guide based on a change in electric conductivity which occurs on contact with the user's skin.

7. A method for supporting a skin treatment using ultrasound, comprising:
determining an ultrasonic treatment map of at least one of a treatment intensity, a treatment number and a treatment depth for at least one skin section of a user with reference to skin condition information including information about at least one of moisture content and wrinkles of the user's skin;
providing the user with a guide for treatment based on the determined ultrasonic treatment map; and
monitoring whether the user follows the guide with reference to treatment action information of the user according to the provided guide.

8. A non-transitory computer-readable storage medium that stores a computer program for executing the method of claim 7.
